SEQUENCE LISTING (17 sequences)

SEQ ID NO: 1 - DNA, 1512 bp, Clostridium thermocellum
  source 1..1512: /mol_type=DNA /organism=Clostridium thermocellum
atgaaaaaagccagaatgaccgttgacaaagattataaaattgccgagatcgacaagcgtatctacggctcttttgtagaacatttgggaagggccgtatatgacggattgtatcagcctggaaattccaaatcggacgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccaggttgggattaatgaatttgcaaaatggtgcaaaaaagtaaatgctgagataatgatggcagtgaaccttggcaccagagggatttcggatgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccaaagacatgcccacttttccccaatgggaagcaacagttctggattatgcttatgattatgtggattatatatcattgcatcagtattatgggaataaagaaaatgacacagctgattttttggcaaaatccgatgatttggatgattttatacgttctgtcattgccacttgtgattatataaaagcaaagaaaagaagcaagaaggatatatacctaagttttgatgaatggaatgtatggtatcactcaaataatgaagatgcaaacattatgcagaacgaaccatggagaatagcgcctcctttactggaggatatatatacgtttgaagatgcgttacttgtcggtttgatgctaattacccttatgaaacacgccgatagaataaaaattgcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgaagatgttaccgatattgaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 2 - DNA, 1512 bp, artificial sequences
  source 1..1512: /mol_type=DNA /note=M12 mutant /organism=artificial sequences
atgaaaaaagccagaatgaccgttgacaaagattataaaattgccgagatcgacaagcgtatctacggctcttttgtagaacatttgggaagggccgtatatgacggattgtatcagcctggaaattccaaatcggacgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccaggttgggattaatgaatttgcaaaatggtgcaaaaaagtaaatgctgagataatgatggcagtgaaccttggcaccagagggatttcggatgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccaaagacatgcccanttttcccnaatgggaagcaacagttctggattatgcttatgattatgtggattatanntcattgcatcagtattatgggaataaagaaaatgacacagctgattttttggcaaaatccgatgatttggatgattttatacgttctgtcattgccacttgtgattatataaaagcaaagaanagaagcaagaaggatatatacctaagttttgatgaatggaatgtatggtatcactcaaataatgaagatgcaaacattatgcagaacgaaccatggagaatancgcctcctttactggangatatatatacgtttgaagatgcgttacttgtcggtttgatgctaattacccttatgaaacacgccgatagaataaaaattgcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgaagatgttaccgatattgaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 3 - DNA, 1512 bp, artificial sequences
  source 1..1512: /mol_type=DNA /note=M20 mutant /organism=artificial sequences
atgaaaaaagccagaatgaccgttgacaaagattataaaattgccgagatcgacaagcgtatctacggctcttttgtagaacatttgggaagggccgtatatgacggattgtatcagcctggaaattccaaatcggacgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccaggttgggattaatgaatttgcaaaatggtgcaaaaaagtaaatgctgaaataatgatggcagtgaaccttggcaccagagggatttcggatgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccanagacatgcccacttttccccaatgggaagcaacagttctggattatgcttatgattatgtggattatatatcattgcatcagtattatgggaataaagaaaatgacacagctgattttttggcaaaatccgatgatttggatgattttatacgttctgtcattgccacttgtgattatataaaagcaaagaaaagaagcaagaaggatatatacctaagttttgatgaatggaatgtanggtatcactnaaataangaagatgcaaacattatgcagaacgaaccatgnagaatagcgcctcctttactggaggatatatatacgtttgaagatgcnttacttgtcggtttgatgctaattacccttatgaaacacgccgatagaataaaaattgcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgaagatgttaccgatattgaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 4 - DNA, 1512 bp, artificial sequences
  source 1..1512: /mol_type=DNA /note= M22 mutant /organism= artificial sequences
atgaaaaaagccagaatgaccgttgacaaagattataaaattgccgagatcgacaagcgtatctacggctcttttgtagaacatttgggaagggccgtatatgacggattgtatcagcctggaaattccaaatcggacgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccaggttgggattaatgaatttgcaaaatggtgcaaaaaagtaaatgctgagataatgatggcagtgaaccttggcaccagagggatttcggatgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccaaagacatgcccacttttccccaatgggaagcaacagttctggattatgcttaggattatnnggattnnatatcattgcatcnntattatgggaataaagaanatgacacagctgattttttggcaaaatccgatgatttggatgattttatacgttctgtcattgccacttgtgattatataaaagcaaagaaaagaagcaagaaggatatatacctaagttttgatgaatggaatgtatggnatcactcaaataatgaagatgcaaacnttatgcagaacgaaccatggagaatagcgcctcctttactggaggatatatatacgtttgaagatgcgttacttgtcggtttgatgctaattacccttatgaaacacgccgatagaataaaaattgcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgaagatgttaccgatattgaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 5 - DNA, 1512 bp, artificial sequences
  source 1..1512: /mol_type=DNA /note=M57 mutant /organism=artificial sequences
atgaaaaaagccagaatgaccgttgacaaagattataaaattgccgagatcgacaagcgtatctacggctcttttgtagaacatttgggaagggccgtatatgacggattgtatcagcctggaaattccaaatcggatgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccagcttgggattaatgaatttgcaaaagggggcaaaaaagtaaatgctgagataatgatggcagtgaaccttggcaccagagggatttcggatgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccaaagacatgcccacttttccccaatgggaagcaacagttctggattatgcttatgattatgtggattatatatcattgcatnagtattatgggaataaagaaaatgacncagctgattttttggcaaaatccgatgatttggatgattttatacgtactgtcattgccacctgtgattatataaaagcaaagaaaagaagcaagaaggatatatacctaagttttgatgaatggaatgtatggtatcactcaaataatgaagatgcaaacattatgcagaacgaaccatggagaatagcgcctcctntactggaggatatatatacgtttgaagatgcgttacttgtcggtttgatgctaattacccttatgaaacacgccgatagaataaaaatttcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgacgatgttaccgatatagaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 6 - DNA, 1512 bp, artificial sequences
  source 1..1512: /mol_type=DNA /note=M60 mutant /organism=artificial sequences
atgaaaaaagccagaatgaccgttgacaaagattataaaattgccgagatcgacaagcgtatctacggctcttttgtagaacatttgggaagggccgtatatgacggattgtatcagcctggaaattccaaatcggatgaagacggttttcgtaaagatgttattgaactggtgaaagaattgaatgtgccaattatccgttatccgggaggcaattttgtgtccaattatttctgggaagatggagtcgggccggtagaggataggcccagacgcttggatttggcttggaaaagtatagaacccaaccagcttgggattaatgaatttgcaaaatggtgcaaaaaagtaaatgctgagataatgatggcagtgaaccttggcaccagagggatttcgggtgcatgtaatttgctggaatactgtaatcatccgggtggttcaaaatatagtgacatgagaataaaacatggagtaaaggaacctcacaacataaaggtttggtgtcttggcaatgaaatggacggtccgtggcaggtcgggcataaaacaatggatgagtacggccggattgctgaagagactgcaagggccatgaaaatgattgacccttcaattgagttggttgcctgcggaagttcctccaaagacatgccctcttttccccaatgggaagcaacagttctggattatgcttatgattatgtggattatatatcattgcatcantattatgggaatanagaanatgannnagctgattttttggcanaatccgatgatttggatgattttatacgtactgtcattgccacctgtgattatataaaagcaaagaanagaagcaagaaggatatatacctaagttttgatgaatggaatgnntggnatcactcaaataatgaagatgcaaacattatgcagaacgaaccatggagaatagcgcctcctttactggaggatatatatacgtttgaagatgcgttacttgtcggtttgatgctaattacccttatgaaacacgccgatagaataaaaatttcctgcctggcacagttgattaatgtaattgcgcctattgtgactgaaagaaatggcggggcggcttggaggcagaccatattttatccgtttatgcatgcttcaaaatatggcagaggaatagtacttcaaccggtgattaacagtccgcttcatgatacttcaaaacatgacgatgttaccgatatagaaagtgttgcaatttacaatgaagaaaaagaagaagtcacaatctttgcagttaacagaaatattcatgaggatattgttcttgtatcggatgtcaggggtatgaaagattatcgtctgctggagcatattgtcttggagcatcaagatctaaaaatccgtaatagtgtaaatggtgaggaggtatatccgaaaaattcggataaatcctcatttgatgatggtattttaacgagtatgcttcgcagagcctcttggaatgtaattcggataggtaaataa

SEQ ID NO: 7 - DNA, 20 bp, artificial sequences
  source 1..20: /mol_type=DNA /note=forward T7 Promoter /organism=artificial sequences
tacgactcactataggggaa

SEQ ID NO: 8 - DNA, 23 bp, artificial sequences
  source 1..23: /mol_type=DNA /note=reverse T7 Promoter /organism=artificial sequences
gtgagtcgtattaatttcgcggt

SEQ ID NO: 9 - DNA, 1455 bp, Thermotoga maritima
  source 1..1455: /mol_type= DNA /organism= Thermotoga maritima
atgtcctacaggatagtggtggatccaaaagaagttgtcaagccgattagcagacacatctacggtcatttcacggaacatctgggaaggtgtatctacggcggaatttatgaagaaggttctccgctctccgatgaaaggggtttcagaaaggacgttctggaggctgtaaagaggataaaagttccgaacttgagatggcccggtggaaacttcgtgtcgaactaccactgggaagacggaataggtcccaaagatcagaggcctgttaggttcgatctcgcctggcaacaggaagagacgaatagatttggaacggacgaattcattgagtactgtcgtgagataggagcagaaccttacatcagtataaacatgggaactggaacactcgacgaagctctccactggcttgaatactgcaatggaaagggtaatacctactacgctcaactcagaagaaagtacggtcatccagaaccttacaacgtaaagttctggggaataggcaacgagatgtacggggaatggcaggtaggccacatgacggcggacgaatacgcaagagccgccaaagaatacacgaaatggatgaaggttttcgaccctacaattaaagcgatcgccgtgggctgtgacgaccccatatggaatctcagggttcttcaagaagcaggtgatgtgattgacttcatatcctaccatttctacacagggtccgacgattactacgaaacggtctctacggtttaccttctcaaagaaagactcatcggagtgaaaaagctcattgatatggtggatactgctagaaagagaggtgtcaaaatcgcccttgatgaatggaacgtatggtacagagtgtccgataacaagctcgaagaaccttacgatctcaaagatggtatctttgcatgtggagtgcttgtacttcttcaaaagatgagcgacatagtcccacttgccaatctcgcacagcttgtaaacgcccttggagctatacacaccgagaaagacggtctcattctcacacccgtttacaaggcttttgaactcatcgtgaatcattccggagaaaagcttgtcaagacccatgttgaatcggagacttacaacatagaaggagtcatgttcatcaacaaaatgcctttctctgtcgagaacgcaccgttccttgatgccgccgcttccatctcagaagatggcaagaaacttttcatcgctgttgtaaactacaggaaagaagacgctttgaaggttccaatcagagtggaaggtctgggacagaaaaaagccaccgtttatacactcacaggtccggacgtgaacgcgagaaacaccatggaaaatccgaacgtcgttgatattacctccgaaaccatcaccgttgacaccgaatttgaacacacgtttaaaccattctcttgcagtgtgattgaggtagaattggagtaa

SEQ ID NO: 10 - DNA, 4385 bp, Thermobacillus xylanilyticus
  source 1..4385: /mol_type= DNA /organism= Thermobacillus xylanilyticus
aagcttatgacgccattccggatcttgagcacgaaggccacgccggacaccacgccgatgtgctgaacgcgatccggaccggggggaagccgctgatcggcggcgaggacggacgggggtcgttcgtgcacaacaccgccgtctacaaagttggtgtgacggcggcaggtggtccggttgtcgttctgtgccggaagaaccgcctctacacccgagaaggaattctggcccgggcggtgaagttttacgagaagaaacgctcggtcgaaggttttgcgccatccggcgacattacgaccggcagcgattaccgataacgggtgccgcggcatgccgaagaacaacggtatgccgcggccagtcctaccctcatgaaggttgagtcgtatggggaacccggccgtatgcgacgcccgggagcccgggtttgcggatgtggctgtgtatctcctatcacacaacaatcagcggaggtgcacccggatatggccgaactcaatctggtgggaagttttcaaggacgacttcgacatcggcgcggcggtgacgtcccggacggtggacagcgcggcggatctgctcagggcgcagtacaacagcatcacggcggagaacgagatgaatcccattaatacgcagccgtctgaaggcgttttcaccttcgagcaggcggacaagattgccgatttcgccgcgaagcacggcaagaagctgcgcggacacacgctcgtctggcacaaccagacgccggactggttcttcgaagcgcccggcggcggtcccgccggcaaggagacgctgctccggcgcatgcgcgaccacatccatgcggtggcgggccggtacaaagggcggacctactgttgggatgtcgtgaacgaagcggtcgccgacgaaggcgaacagtggctgcgggcgtcgaaatggcacgacatggtcgggccggaattcatcgtccgcgcgttcgagtacgcgcacgaggccgatcccgacgcgctgctgttctacaacgactacaacgaatgcaatcccgcgaagcgcgacaagatcatccgcctcgtgaaatggctcaaagagcagggcgcgccgatccacggcatcggcatgcaggggcactacaatctggcgtcgccgtccatcgccgaggtgcgcgaggcgatcgagaagtacgccgagctcggcctggtcattcatgtgacggaactggacatgtccgtctacgcctgggatgaccggcgaaccgatctgctcgagccgacggcggaaatggtggagcggcaggcggagctgtacgagcagctgttctcgctttaccgcgaataccgggacgtcatccgcagcgtcacgttctggggcgcggcggacgattatgcgtggctcagctatttcccggtgcgcgggcggcgcaactggccgctgctgttcgacgcgcagcaccggccgaaggaggcgttccggcgcgtcgtgcggacggcgggcgctgaagcttgagctggcggaaaccggcaattgcagccggtctgttccggagaaacggacggaccggctgttgctgttccgcgggcttagattcgtgagcaccgccgcgttacgcccggacggttgtttttgcccttccggagcggaattcgccgggcgtgaggccgatgtacttcttgaactgccgcatgaagtggacgtcgttctcgtatccgcactgtgcggcgatgtcgcggatgctgtcggacgtgtttttcagccggaacatcgcgtattcgaccgggttgtggatgatgtcgttcatgaacgagatgccgaacagttctttgtacagcttctggaaatagggccggctcatgttgacgcggcccgccagatcgccgatcgtccaggcgacggacgggttgctgtatatgtcgctgcgcagcgacacgagcagatcgtaatatttgctgaccggcttgctcggctcgatccggttgcggatgtcgctgagcttcatgaggaggcagcggacggtcgcgtcgatgatctcgtcccggtacagcccgttctggaccagcgcttcctggatttcgctcactttccgcgacacatataccggttcatgcaccttcatcggcgtgtccagcggcagattcagtttccggcagaacgtctcgacatcctcgccgtcaaaatggaaccagtcgtgcaccatcggctgttcgatgtcatggtagtattgccggcttccttttccggtaaatgatgcaggtgttcttgtccgtcaggaagcgctctccctgcgcttcgacgcacatgcgcctgcggaagaacaggaacagataatcgcccgacccgtgcggccggtcgatgacgatgccttccggatggccgacgttgtagccgcaccggttcatctggaacatcgcgccacccccttgaagtcgagagatcagtgattgattatgatagatcatttcgccaatgcgatcaaacctttacggtgattagtgaagacgagagtcgcggggattcgaatatgagagttgcacggcccgcagaagataatctttcagcaaaatgaaggagcgatgaacgtggcaagccgggtagtcgtaaacgccgacagggtgaagggcacgatcaaccgcaacatttacggccatttctcggaacatctcgggcgatgcatctacgaagggctgtgggtcggagaagattcgccgattccgaatacgaacgggatccgaaacgacgtgctcgaggcgctgaagcagatgaaaattccggtcctccgctggccgggcggctgcttcgcggacgaataccattggaaagacggcgtcggcccacgggagaagcggaagcggatggtgaacacgcactggggcggcgtcatcgagaacaaccatttcggcacccatgagttcatgatgctgtgcgagctgctcggctgcgaaccgtacatcagcggcaacgtgggcagcggcaccgtgcaggagatgtccgaatgggtcgagtacattacgttcgacggcgaatcgccgatggcgaactggcgcagggagaacggacgggagaagccgtggcgcatcaagtactggggcgtcggcaacgagaactggggctgcggcggcaacatgcgcgcggaatattacgccgatctgtaccgccagttccagacgtatctgcgcaactacggcgacaacaagctgcacaagattgcgtgcggcgcgaacacggccgactaccattggacggaagtgctgatgaagcaggccgcgccgttcatgcacgggctgtcgctgcactattacaccgttccgggcccctgggagaagaaaggacccgcgaccggcttcacgacggatgaatggtgggtcacgctgaagaaggcgctgttcatggacaggctcgtcacgaagcattcggccatcatggacgtctacgatccggacaagcggatcgatctgatcgtggacgaatggggcacgtggtatgacgtcgaaccgggcacgaatccgggcttcctgtatcagcagaactcgatccgcgacgcgcttgtcgcgggcgcgacgctgcacattttccaccgccattgcgaccgcgtccggatggcgaacatcgcccagctcgtcaacgtgctgcaatccgtcatcctgacggagggcgagcggatgctgctgacgccgacctaccatgtgttcaacatgttcaaggtgcatcaggatgcggaactgctcgatacatgggagtctgtggagcgcacgggtccggaaggcgagctgccgaaggtatcggtctccgcttcccgcgcggcggacggcaagatccacatcagtctgtgcaatctggacttcgagaccggagcaagcgtggacatcgagctgcgcggcctgaacggcggcgtgagcgcaaccggcacgacgctgacgtcgggccgcatcgacgggcacaacacgttcgacgagcccgagcgggtgaaaccggccccgttccgcgacttcaaactcgagggcgggcacctgaacgcatcgctgccgccgatgtccgtgacggtgcttgagctgacggcggggtgagcggcggatgacggcggaacgcccgtgcagaggctgcaggtctgacgcgcacgtgtcggaagaacgcatccgggcgatgctcgccgcgccgatgttcgcggaatcaagcgggctgtgcgtgccggacgacgtctatgagatgcggctggacctctgccggtcctgtccgaagctgattggcggccatacatgcgcggtctgcggctgcatcgttccgatccaaagaattcaaaaagcttctcgagagtacttctagagcggccgcgggcccatcgattttccacccgggtggggtaccaggtaagtgtacccaattcgccctatagtgagtcgtattac

SEQ ID NO: 11 - DNA, 1000 bp, Geobacillus stearothermophilus
  source 1..1000: /mol_type= DNA /organism= Geobacillus stearothermophilus
gatccggaaaagcgcgtcgggctcattgtggatgaatggggaacgtggtatgacgtagaaccgggcacgaatccgggattcttatatcaacaaaatacaatccgtgatgcgcttgtcgcagctctccattttcatattttccataaccattgtgatcgagtgcgaatggcgaatatcgcgcaaacggttaacgtcttgcaagcggtcattttgacaagaggggaacaaatgattctcacgcctacgtatcatgtgtttgacatgtataaggttcaccaagatgccgagttgttggcaatagagtcttcatgtgccgagtatgagcatcgcggggaaacgttgccgcaagtaagcatcactgcatccaagaacggggaaggaactgtccatatcagtttatgcaatattgatcatcgaaatgaggcggtagtagatttagagtttcgtggagacagcctcgcacgcaagcaagtattcggcacgatgttaacagcgaatgagatgaatgcccataatacatttgaaaagccggatgcagtgaagcccgaaccgttccgtcaaatagagctcggccaacatacactgatggccaaacttccgcccatgtctgtcgtcactttagcgattgttgaaaataaagcggctttgattgaacgacaatgaagacaaaggcggcggaaaatatggaagaaaaaaattggtgccaagactgcgcgggcagcgtcatcgttacacgggaaatgatggatccgctcattgaataaattgatcgttcagcactcgtttccgatgacgtatatgagaaaaggatcaatatatgcagaagttgttcctctctccaatatggcacaacttgcgcctatagcgggtatcttgtccattaccaggcaaaatggaggcgggaatgttgcccgtttccgggaagggcgaaatggggaagggaagaatagattgacaagttgataaaaaatagaaaggaagagtggcagatggctacgaa

SEQ ID NO: 12 - DNA, 1914 bp, Aspergillus oryzae
  source 1..1914: /mol_type= DNA /organism= Aspergillus oryzae
atgctacgcaaacgttttcttctgcccctcctggcagcttgcggtgcagcggttgagatctccgttgcatcgtctggtggtaatgccactagtggtctacaatatggtatcatggaagaggaaatcaactactgtggtgatggaggtctctatgcggagctgattcgcaatcgtgcattccagggaggcgagaaatacccttccaacctggatgcttggatcccaatcgacggatcagcactgtctctgaagaacctcagtcaacccctgtcgtcggcattgcccacatcagtcaatgtcaaaggaacagcaggaaaggccggtcttaccaacttaggttggtggggcatcgacgtgagagagcagacgtataccggctccttctacgtgaagggtgcctacaacggaacattcacagcttccctacaatccaataagacgggcgaggtatatgccagcgctgtcattgtgtccaagagcgctcgcggcgaatggacacagcataactttactttgacccccaccaaggctgcctcaaacacccagaatacgttctctattaccttcgatgcttctaacactgttgatggctccctggacttcaaccttatcagcctgttccctcctacatataatgaccgtcccaatgggcttcgcagggatttgatgcaagctatggccgacttcggacctaaatttctgcggttcccaggtggcaacaacctcgaaggcgatacccttgatggcagatggaaatggaacgaaaccattggacctctcaaggaccgcccaggccgtgcaacaacttggtcctaccaagaaacccacggattaggtctcgtcgaatacatggaatggtgtgaagaccttggagtcgagcccatccttgctgtatggggtggattcgctctgaacggcgacgcaattcccgaatccgagctcggtacatatgtgcaagatgccctcgacgagctggaatttctcaccggctccgtggacaccgaatacggtgccctccgcgcgtccctcggccacccagacccatggacagtcaagtatgtcgaagtcggcaacgaagataacctaaacgacggattggattcatacaaatcctatcgtttccaggcattctacgacgccatcaaggagaagtacccagacatcaccgtcctagcatcgaccgtagaaatcgacttccccggcgatgcaggcggtgactatcacctctacgatacgccagacaacttcgtcgagaagttcaactacttcgaccagtacagccccgaccatccgatcctccttggcgaaatcgccgcgattcagctcaacggacgcgaaatcgtctggggcaactccagccatttctcacagtacccatggtggatcggcagtgtcgcagaaggggtcttcctcatcggcgccgaaagaaacgccgacaaagtccttggaacaacatacgcccccttcatgatgaacctcgacaattaccaatggtcccctaccttcctcgccttcaactcgaacccagacgagacggcacgctcgaccagttggtacctttacgatctcttctcccacaactccttcacacacaccctccccacaacatcaaattccagcttcggacctctctactacgtcgctggtgttgataacacgagcaattctcacatcttcaaggcagctgtttacaacagcaccgccgacgtgcccgtttctctcaccttcgacggtgtcaaggccggtaccagcgcaagccttactgtgctcaccgccgcggatccactgggtatgaacgaagtaggagctgctaatatcgtcgataagaagacctccaccgtgacggcaggtgtcaacggcgtgtttgacttcagtctgccaaatctcagtgtggcagttttgaagacggagtag

SEQ ID NO: 13 - DNA, 1000 bp, Bacillus subtilis
  source 1..1000: /mol_type= DNA /organism= Bacillus subtilis
atctttttcggctttttttagtatccacagaggttatcgacaacattttcacattaccaacccctgtggacaaggttttttcaacaggttgtccgctttgtggataagattgtgacaaccattgcaagctctcgtttattttggtattatatttgtgttttaactcttgattactaatcctacctttcctctttatccacaaagtgtggataagttgtggattgatttcacacagcttgtgtagaaggttgtccacaagttgtgaaatttgtcgaaaagctatttatctactatattatatgttttcaacatttaatgtgtacgaatggtaagcgccatttgctctttttttgtgttctataacagagaaagacgccattttctaagaaaaggagggacgtgccggaagatggaaaatatattagacctgtggaaccaagcccttgctcaaatcgaaaaaaagttgagcaaaccgagttttgagacttggatgaagtcaaccaaagcccactcactgcaaggcgatacattaacaatcacggctcccaatgaatttgccagagactggctggagtccagatacttgcatctgattgcagatactatatatgaattaaccggggaagaattgagcattaagtttgtcattcctcaaaatcaagatgttgaggactttatgccgaaaccgcaagtcaaaaaagcggtcaaagaagatacatctgattttcctcaaaatatgctcaatccaaaatatacttttgatacttttgtcatcggatctggaaaccgatttgcacatgctgcttccctcgcagtagcggaagcgcccgcgaaagcttacaaccctttatttatctatgggggcgtcggcttagggaaaacacacttaatgcatgcgatcggccattatgtaatagatcataatccttctgccaaagtggtttatctgtcttctgagaaatttacaaacgaattcatcaactctatccgagataataaa

SEQ ID NO: 14 - DNA, 1980 bp, Cellvibrio japonicus
  source 1..1980: /mol_type= DNA /organism= Cellvibrio japonicus
ccgatgaattcacagcgttcaatcgcgacatcgagcaggtctatcagcgtccgaccctgatagttaaatagtcttttctcttgactgattcgccgccccagcggttgccggggcggcgaatttgtcctgcttaactgatacccgggataaccgtaaaaataggttaaattgtaggacaataatatcttatggcctgtattctcccacggtttcacgtttgataaaaattcccgataacaacaaaggagaatcccatgcgccgtttgaaacccctcatcgccgcacttggcttgagttttagcatcggcagcttcgccaatacccacatcaccatagataccaccaaatccggtccggtgattaacaagaacatctatggccaatttgccgagcatctgggacgaggtatttatgaaggactgtgggttggtccggagtccggcatccccaacacccgcggttggcgcaatgacgtggtgggtgcgctcaaggatattaacgtgcccctggtgcgttggcccggtggctgttttgcggatgaataccactggcgtgatggcataggcccgcgcgaccagcgcccggtgaaggtcaataccaactgggggggcgtggaagaggataatgccgtggggacccacgagttttttgacctggtggagattctgggggctgaagcctatgtgaatggcaacctgggcaccggaacgccccaggaaatggccgaatggctggaatatatgacggcggagggtaaatcaaccctggcggaattgcgccgtaaaaatggccgcgataaacccttccaggtccagtattttgcaattggtaatgaggcgtggggctgcggtggcaatatgacgcccgagtactacaccaacctgtacaaccactacgcgactttcctgaaggcccctgcccacaatgcacccaaattgattgccagcggcggccataccgaagacaccagctgggctgcccacctgaccgccaatgtcaaacccaactggagtttgaaaatggatgccgttagtttccactactacaccctgcccacgggtaaatgggataagaaaggtgcggccatcggcttcccggaggcggagtggatgtctacgctggtgaataccctgcgcatggatgactttatcgtcaacaacaaaaaagtcatggataaaaacgatcccgagaaaaaagtgggcttctatgtggacgagtggggcacctggtacgacgtggaagcgggcgagaatcccggtttcctttaccagcagaacagtttgcgcgatgcggtggtagcggcacttaacttcaatattttccacaagcatgcagatcgggtgcacatgaccaacattgcgcagatggtcaatgtgttgcaggccatgattttgaccgataaggaaaaaatgatcctgacgcccacctactatgcgtacaagatgtatgtacccttccaggatgcaacatcgctgcctgtcagcttgaaaaaagtgtcccaataccgtttggggaaatcctccgtgccggcgatcagcgcttcagccgcgcgcggtaaggatggcaaagtgtacctggccctggtcaatgccaatcccaaccaggccgagacggtagcgcttgcattacctggtgtaactgccagcggggtgagtggccagttgctgacagcgacagcgatggatgcgcataacacctttgccaatcccaatgcgatcaagccggtgagttattctgcgaaagccgtgaatggcaaattgtcgctggagctgcctgctaaatcggtcgttgtggttgcggtggaatagggcgcgtttttcagatggaaaccggagtccggtcgttggtgttaataccgacgaccgggttttggttaagtgcgagtgacaataattaaaacaaatcctcaatcgacaaatagcgctcgccggtatcgtaactgaataccagcacgcggctaccgggtttcatatcagcttg

SEQ ID NO: 15 - DNA, 2132 bp, Streptomyces avermitilis
  source 1..2132: /mol_type= DNA /organism= Streptomyces avermitilis
atgtcacccacccgcacccgctggagactcggactcacagccgccgcgttactggtggcctcggtcggcgttcccgcgcccgcccacgccgaagacatcaccgactacgcgatcgccgtcgatcccaagggatcgggggcgaagatcgacgacaccatgtacggggtgttcttcgaggacatcaacagggccgccgacggcgggctctacgcggaactcgtgcagaacaggtcgttcgagtacgcgaccgccgacaacacgtcgtacaccccgctcacctcctggaacacctccggtaccgcggacgtcgtcagcgacgacggacggctcaatgcccgcaaccgcagctatctggccctcggcggcgactcctcggtcaccaactccggctacaacaccggcatcgcggtcgagagcggcaaggtctacgacttctccgtctgggcccgcgccgaccaggccgaccccctcagcgtgaccctgcacgacacggacggtgacttggcccgggcccgccgcgtcaccgtgcgcggcggctgggccaagtacacggccaggttcaccgccggccgcaccagcaccaccggccggctcaccgtcgccgccgccggcgccgtcgcgctcgacatggtctcgctcatccctcatgacacctacatggggcacgggctgcgcaaggacctcgccgagaagatcgccgccctgcaccccggcttcgtccgcttccccggcggctgtctcgtcaacaccggttcgatgaggggatacgagaggcgtcggggtacgagcgcaagcggtcgtaccagtggaaggacaccatcggacccgtcgagcagcgcgccaccaacgccaacttctggggctacaaccagagttacgggctcggctactacgagtacttccagttcgccgaggacacgggcgcgatgccgctgcccgtggtgcccgccctcgtcaccggctgcggccagaacaaggccgtcgacgacgacgctctcctcgagcgccacatccaggacacgctggacctcatcgagttcgccaacggcccggtgaccagcgaatggggcaggaagcgcgccaggatgggccatcccgagcccttccacctcacccacctcgaggtgggcaacgaggagaacctccccgacgagttcttcgcccgcttcacgcggttccgggccgccatcgaggcgaagtaccccgacgtcacggtcatctccaacgccggcccggacgacagcggtccgaccttcgacaccgcctggaagctcaaccgtgacgcggacgtcgacatggtcgacgagcactactacaacagcccccagtggttcctccagaacaacgaccgctacgacgcgtacgacaggaacggccccaaggtgtttctcggcgagtacgcgtcgggcggcaacaccttcaagaacgccctggccgaagccgcgtacatgaccggtctggagcgcaacgcggacgtcgtcaagctggcctcgtacgcaccgctgctcgccaacgaggactatgtgcagtggcgccccgacatgatctggttcgacaaccacgcctcctggggctccgccgactacgaggtccagaagctcttcatgacgaataccggcgaccgggtcgtgcccagcaccgcgagcggcacgcccgccctgagcgggccgatctccggtgcggtcggcctgtcgacgtgggcgaccaccgccgcgtacgacgatgtgcgggtcaccggcgaggacgggaccacgctgctcggcgacgacttcagcggtgacgcctcgcggtggacgcacacgggcggcggaagctggagcgtcgaggacgggcagtacgtgcagagcgacgtggccgccgagaacaccatggtctcggcgggcgacccggcctggcacgactacgacctgcgggtgaaggccaccaagaagtccggcaaggagggcttcctcgtcgcgttcggcgtcaaggacaccggcacctacacgctcgtcgccgcgcccgacgcggtgaactcgcggacggcgacgcctgtcgcaccggtgacgtccgtcttggagggggtggccgacaggttcacgtataccttcccggccaactccgtgacgttcctgcggatcaggcagaggtag

SEQ ID NO: 16 - DNA, 2800 bp, Aspergillus kawachii
  source 1..2800: /mol_type= DNA /organism= Aspergillus kawachii
tagtgggtccggtaagctagacaattcaaacgcagccattcacagatttaattcaaagacaaggctggatagtatgctgatagagttcaattagcatgaccacatggtatgaagaacatgggttcccctcactgcaccggttaaagccaccccccttcccaaaagtaatgatcgtacgacgggcacgggtcggttgttatgtctactttatgctatttcgactatacttattgcaaagcggaattcgttccgatcaacatgcagctatccagagacattcatttcttaatatccggggtaatggcatccaaattgtagctcccaaaagtggcccattactcgaaaacacagtgcctccggataagcattcagcaaccctctacaaaacaaacttccacaggaatgcggcacctccatcccctccacaagtaccgaggcttgatccaggaggaacagatccctgaagaaacgtgacgaatcgtgatgagcgggcaacacagtttagtctcctccccccgcatcccctccgggcaaaaccccggcggcactagcattagccctaggcacggacaatgccattgtcaagccttcaggaactaaatcaactaaatcactcccatcctgcggggtacatactccacgacgccctgcacgcccgccggaataccgtcgtcgcttgacatcaggtggaacaacgcattcctgctgcctcgaaagtgagccttttaaccgaccggttacttgacaatgtatacaggatgtcccaatattggaggaaaatccccgacccctgaagccacacatgtaattagacttcccatatcatgcactccatgcatgggcacttcactgcagtgaagaatataaataagccccccttctcaccatagtagcatgacttctttcaccaagcggcctatcctactactgtctgagtcctctcctagcccagaccttcctgagcaggcagttatccatccaccaccatgttctctcgccgaaacctgctcgccttagggctagccgccaccgtcagcgccggcccctgtgacatctacgaagccggcgacactccctgcgtagccgcacacagcaccacccgcgccctatacagctccttcagcggcgccctctaccagctccagcgcggctccgatgataccaccaccaccatctccccgctcacagccggcggcatcgccgatgcctccgcgcaagacaccttctgcgccaacaccacctgcctgatcaccatcatctacgatcaatctggcaacggcaaccacctgacccaagcacccccaggcggcttcgacggcccagacaccgacggctacgacaacctggccagcgccatcggcgcccccgtcaccctgaacggccaaaaggcctacggagtcttcatgtcccccggcacgggttaccgcaacaacgaagccacgggcaccgccaccggcgacgaggccgaaggcatgtacgccgtcctggacggcacgcactacaacgacgcctgctgctttgactacggcaatgccgaaaccagcagcaccgacaccggcgccggccacatggaggccatctacctcggtaacagcaccacctggggctacggtgccggcgatggcccctggatcatggtcgacatggagaacaacctcttctctggtgccgacgagggatataactccggtgatccgtctatctcttaccgcttcgtcactgccgcagttaagggcggcgctgataagtgggctattcgcggcgctaatgctgcctctggctccctgtccacgtactatagcggtgcccgtccggattactccggctataaccccatgagcaaggagggtgctatcatcctgggtattggtggtgataacagcaacggcgcccaaggtaccttctacgagggtgtcatgacctccggatatccctcggatgataccgagaactccgttcaggagaacatcgtcgctgctaaatacgtcgtcggctcgctggttagcggtccgtcgtttacctccggagaggttgtttcgctgcgtgtcacgaccccggggtacacgacgaggtatatcgcgcatactgacacgactgtgaacacacaggttgtggatgatgatagttccacgacgttgaaggaggaggctagctggacggttgtcactggtttggctaacagccagtgcttctcgttcgagtcggttgatacccctggtagctatatccggcattataactttgagttgctgcttaatgccaatgatggcacgaagcagttccatgaggatgctactttctgtcctcaggcggcgttgaatggtgagggtacttcgttgcgctcgtggagttacccgacgaggtatttcagacattatgagaatgttctctatgctgctagcaatggtggtgtgcagacgtttgactcgaagacgtcgtttaataatgatgttagctttgagattgagacggcgtttgcttcgtgaggggttattaggggttggttgatcgggtgggtatgagggatcgtggtttgtaaataggttatgtttgctatatagcatatgaatactatacgatggtatactttaatgtggattgtgtctatgtaatctacagaagcatcagacccttgcctacatgcaacacaatatcacaacattgtatccgtggagccgtcacggactaatcagctgcgagatggttcttatgcggtgctagttactgtgattggggataagttcaagcgtagttgtctttccttcacaaaatgcatgtggatattgtatgtcacggtagc

SEQ ID NO: 17 - DNA, 1971 bp, Aspergillus terreus
  source 1..1971: /mol_type=DNA /organism=Aspergillus terreus
atgtgggcgtgtcttcttgccaggctcattctcggagttctcaccctgtctgggttgacatgggcgcgagcggacccagggtcatcgacgagggaggatgtcgagaagaaaaataatagtgcgccgattaccttgacggttgcaaaggagggcggaaaccagtccagttccctcctgtgggggatcatgtttgaggaaatggatcaatcgggtatgatccctcaaccaccaccaccacctctctcctcccaatgtaaaattgctgaaaacgcgcaggagatggcgggattcacggtcaactactgcggaacaacggacaacctcaatccggtcagccatgccatcacctcctcgctccaagtgcaggtgatgccgtgggagtcaaagctggtggggttctccaactcgggctataatgggatccctgtgatgaacgacacgtatcgatgtgaattctggatgaagggtgtttattcggggcccatcaccctgcagctcgtcgggtcagccagtgggatcgtttatgccaatcacaacatcacagtgaacagtacgtttggggaattcactcgctacgaaaccttcttcaactcgactgcttctccggacggcaataatgagtggcgtctgctgtttgatggttcaagagtggacgggggatcgctcaactttgggctaccgcagctattcccacccacctatcattcaagggtgaatggtctacgtaaagacttggcgactacactggaacaaatcaagccgtcctttctccggtttcccggtggaaataacatagagggaatccaaatcgacaacagatggatctggaaccagaccattgggccagtcgttgaacgtcctggaagacagatactaacaatccaggtgattggcattatccaaacacggacgctctcggtgagtctagccttggacagtgagcaggatcaggcactgacatgtgcaggactcgatgaatacctctggtggtgtgaggacatgggaatggcccctgttctagccgtgtgggatggcaaatcgtatggaggaatcctttctggttccgatctacagcctttcctcgacgatatcatgaacgagatggagtacctctttggtcctcccaacagcacctacggcagcatgagggcaaagaacggtcgcaaagagccctggttggtagagtatatcgagattggcaacgaggatgatttcaccggcggatgtccaacataccccgatcgctttactcaggtctatgatgctattcacgagaaatatcccaacgtcactctgatcgcatctaccaatgatttcgattgtttgccggagtctcccccggacggaatcatgtacgatttccactactaccgcaagccggacgatctggttgcgatgttcaattactgggataaccagccacgctcccagcccatcatggtcggtgagtttggttgtcgcaataccagcgacgcccacggagtctactggtcttccatgaaaatgagctgtagtgaggccgcacatatgatcggtttggaacgaaacagtgatgtggtgaagatggcggcgtatgcgccgttattgcagcattttggctatacacagtggtcgccaacgctctatgggttcgactcgagcccggggtcgataaccccctctacctcatactacgtccagcagatgttttcaaccaaccgagggtctacgattctccccgtcaactcgaccgcaggctttggaccgctgtactgggttgcctcccacacaaacgacacatactacaccgtctatgtggaggtgccagacacgaagtctggcaaactggagacgctttcgggtccgagagacgactacaacattcctcataatgtgaccatcaagccggtcaccaagaacgtgaccgtatctcatgggaactataccatcgagatgaccccatggtctgttgcggtactggtggtctcgtga

(19) United States
(12) Patent Application Publication
(10) Pub. No.: US 2015/0125888 A1
(43) Pub. Date: May 7, 2015

(54) ONE STEP ENZYMATIC PROCESS FOR PRODUCING ALKYL FURANOSIDES

(71) Applicant: ECOLE NATIONALE SUPÉRIEURE DE CHIMIE DE RENNES (ENSCR), Rennes Cedex 7 (FR)

(72) Inventors: Richard Daniellou, Sens de Bretagne (FR); Caroline Nugier-Chauvin, Saint-Gregoire (FR); Vincent Ferrieres, Gahard (FR); Alize Pennec, Rennes (FR); Ilona Chlubnova, Praha 7 (CZ)

(73) Assignees: ECOLE NATIONALE SUPERIEURE DE CHIMIE DE RENNES (ENSCR), Rennes Cedex 7 (FR); CENTRE NATIOANAL DE LA RECHERCHE SCIENTIFIQUE (CNRS), Paris (FR)

(21) Appl. No.: 14/396,448

(22) PCT Filed: Apr. 19, 2013
(86) PCT No.: PCT/EP2013/058187

§ 371 Date: Oct. 23, 2014

Related U.S. Application Data
(60) Provisional application No. 61/636,883, filed on Apr. 23, 2012.

(51) Int. Cl. C12P 19/44; C12Q 1/34; C12N 9/24

(52) U.S. Cl. CPC C12P 19/44; C12N 9/2402; C12Q 1/34; C12Y 302/01055
USPC 435/18; 435/74; 435/200; 506/11

(57) ABSTRACT

A process for enzymatically converting a furanoside substrate in a product of interest, includes contacting the substrate with an enzyme in presence of an alcohol acceptor, wherein the enzyme is preferably Araf51, and wherein the product is preferably an alkyl furanoside. The mutant Araf51 enzyme showing improved transglycosylation activity in comparison with the native wild-type (wt) Araf51 enzyme, and a method for screening the mutants are also described.

### FIELD OF INVENTION

The present invention relates to innovative and eco-friendly enzymatic syntheses of structurally well-defined alkyl furanosides from polysaccharide raw material. The present invention also relates to native and/or mutant enzymes for implementing said syntheses.

### BACKGROUND OF INVENTION

Glycofuranosidic compounds present a large diversity of properties and potential uses depending on the nature of the alkyl chain as well as the glycofuranosyl entity:
Butyl furanoside could act as a chemical building block for further derivatization essentially for industrial preparation of alkyl polyglycoside (APG);
Octyl-furanoside, as an amphiphilic molecule, could exhibit interesting surfactant properties, for instance in the field of cosmetics or detergence;
Furanosyl-containing glycoconjugates are involved in some pathogenic microorganisms responsible for parasitic and neglected diseases. Some of these alkyl furanosides reveal biological activities as immunostimulating agents and anti-parasitic drugs;
Alkyl furanoside consists in a monomeric entity that could be easily incorporated into biodegradable materials.

The need for improved and bioresource-adapted conversion technology remains a challenge for the biorefinery.

Arabinofuranosyl hydrolase Araf51 is naturally involved in the hydrolysis of natural polysaccharides from lignocellulosic biomass (Taylor et al, Biochem. J., 2006, 395, 31-37). The Inventors herein show that this enzyme can also catalyze the transglycosylation of furanosyl residues to diverse acceptors including alcohols. As an example, arabinofuranosyl hydrolase Araf51 may catalyze the transfer of an arabinofuranosyl entity to various alcohol acceptors (scheme 1).

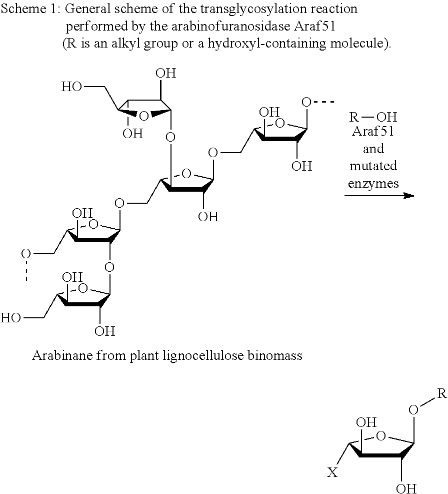

Moreover, the Inventors identified mutations of the Araf51 enzymes, showing improved catalytic efficiency of the transglycosylation reaction.

The major challenge faced by the Inventors consisted in the use of natural arabinan and arabinoxylan polymers as glycofuranosyl donors. In fact, despite being an extremely large resource from the plant biomass, natural arabinan and arabinoxylan polymers are hardly depolymerised and thus still not much used as renewable carbon sources. More generally the industrial fermentation of pentoses from hemicelluloses has not yet been achieved in a cost efficient way.

### SUMMARY

The present invention thus relates to a process for enzymatically converting a substrate in a product of interest, comprising contacting said substrate with an enzyme in presence of an alcohol acceptor, wherein said substrate preferably is a furanosyl-containing polysaccharide substrate, wherein said product of interest preferably is a furanoside; the enzyme preferably is an Araf51 enzyme, which may be native or mutant.

The present invention also relates to a mutant Araf51 enzyme showing improved transglycosylation activity in comparison with the native wild-type (wt) Araf51 enzyme, wherein said mutant enzyme presents at least one of the following features:
no inhibition in presence of alcohol acceptors;
increased kinetic conversion rate; and/or
molar conversion yield of more than 30%.

The present invention also relates to a method for screening mutant Araf51 enzyme showing improved transglycosylation of a selection substrate activity in comparison with the native wild-type (wt) Araf51 enzyme.

The present invention also relates to a process for producing alkyl furanosides comprising contacting a polysaccharide with a native Araf51 enzyme or a mutant Araf51 enzyme showing improved transglycosylation activity in comparison with the native wild-type (wt) Araf51 enzyme, in presence of an alcohol acceptor.

### DEFINITIONS

In the sense of the present invention, the following terms have the following meanings:

“About” preceding a figure means plus or less 10% of the value of said figure.

“Transglycosylation” refers to a chemical reaction wherein sugar moieties are transferred from activated donor molecules to specific acceptors, forming a specific glycosidic bond.

“Alkyl”: refers to any saturated linear or branched hydrocarbon moiety, with 1 to 12 carbon atoms, preferably 1 to 6 carbon atoms, and more preferably methyl, ethyl, propyl, isopropyl, n-butyl, sec-butyl, isobutyl and tert-butyl.

“Alkenyl”: refers to any linear or branched hydrocarbon moiety having at least one double bond, of 2 to 12 carbon atoms, and preferably 3 to 6 carbon atoms.

“Allylic”: refers to an organic moiety with the structural formula R₁R₂C═CR₃—CR₄R₅R₆. In one embodiment, each of of R₁ to R₆ is independently H, alkyl, or alkenyl. In one embodiment, each of R₁ to R₆ is H.

“Furanoside”: refers to the furanose form of a glycoside, wherein a glycoside is a molecule in which a sugar group (the glycone) is bound to a non-sugar group (the corresponding aglycone), such as for example an alkyl or an alkenyl group or an allylic group. The term furanoside in the meaning of this invention thus encompasses alkyl furanoside, alkenyl furanoside and allylic furanoside.

“Alkyl furanoside”: refers to any sugar in the furanose form linked with an alkyl group.

“Alkenyl furanoside”: refers to any sugar in the furanose form linked with an alkenyl group.

“Allylic furanoside”: refers to any sugar in the furanose form linked with an allylic group.

“Activated furanoside”: refers to furanoside bearing a good leaving group as an aglycon.

“Lignocellulosic biomass”: refers to plant biomass that is composed of cellulose, hemicellulose, and lignin. Lignocellulosic biomass may correspond to agricultural residues, dedicated energy crops, wood residues, and municipal paper waste.

“Aliphatic alcohols”: refers to organic compounds containing one or more hydroxyl groups [—OH] attached to an alkyl radical.

“Allylic alcohol”: refers to an organic compound with the structural formula. R₁R₂C═CR₃—CR₄R₅OH. In one embodiment, each of of R₁ to R₅ is independently H, alkyl, or alkenyl. In one embodiment, each of of R₁ to R₅ is H, and the allylic alcohol is prop-2-en-1-ol.

“Alkenic alcohols”: refers to organic compounds containing one or more hydroxyl groups [—OH] attached to an alkenyl radical.

“Diastereoselective”: refers to an enzyme having a preference for the formation of one or more than one diastereomer over the other in an organic reaction.

### DETAILED DESCRIPTION

### Enzymatic Process

A first object of the invention is a process for enzymatically converting a substrate in a product of interest, comprising contacting said substrate with an enzyme in presence of an alcohol acceptor.

In one embodiment, the process of the invention is a one step process.

In one embodiment of the invention, the enzymatic conversion is a transglycosylation, preferably a transglycosylation of furanosyl residues to alcohol acceptors.

In one embodiment of the invention, the enzyme is an arabinofuranosidase, preferably selected from the group comprising proteins of the GH51 family, such as, for example, Araf51 GH51 from Clostridium thermocellum (encoded by the nucleotide sequence SEQ ID NO: 1), Tm-AFase GH51 from Thermotoga maritima (SEQ ID NO: 9), AbfD3 GH51 from Thermobacillus xylaniliticus (SEQ ID NO: 10), AbfAT-6 GH51 from Geobacillus stearothermophilus (SEQ ID NO: 11), AbfA GH51 from Aspergillus oryzae (SEQ ID NO: 12); GH 43 from Bacillus subtilis (SEQ ID NO: 13); Abf51A from Cellvibrio japonicus (SEQ ID NO: 14); CBM42 GH42 from Streptomyces avermitilis (SEQ ID NO: 15); AkabfB GH54 Aspergillus kawachii (SEQ ID NO: 16); and a-ara pI from Aspergillus terreus (SEQ ID NO: 17).

In one embodiment of the invention, the enzyme is an Araf51 enzyme, preferably the Araf51 enzyme from Clostridium thermocellum (SEQ ID NO: 1). In a first embodiment of the invention, the Araf51 enzyme is a native Araf51 enzyme. In a second embodiment of the invention, the Araf51 enzyme is a mutant Araf51 enzyme as described below.

In one embodiment, the mutant Araf51 enzyme presents at least one of the following features:
no inhibition in presence of alcohol acceptors;
increased kinetic conversion rate; and/or
molar conversion yield of more than 30%.

In a preferred embodiment, the mutant Araf51 enzyme is encoded by a nucleotide sequence selected from the group consisting of SEQ ID NO: 2 (M12 mutant), SEQ ID NO: 3 (M20 mutant), SEQ ID NO: 4 (M22 mutant), SEQ ID NO: 5 (M57 mutant) and SEQ ID NO: 6 (M60 mutant).

In one embodiment of the invention, the substrate is a furanosyl substrate. In a first embodiment of the invention, the substrate is a natural substrate, preferably a natural furanosyl-containing polysaccharide raw material, more preferably is arabinoxylan or arabinan, such as, for example, branched or debranched arabinan.

In a second embodiment wherein the enzyme is a mutant Araf51 enzyme and the substrate is the selection substrate of the mutant Araf51 enzyme, preferably said selection substrate is p-nitrophenyl α-L-arabinofuranoside.

Examples of polysaccharide raw materials used as substrates include, but are not limited to natural arabinan polymers, natural arabinoxylan polymers, pentoses from hemicellulose, branched arabinan, debranched arabinan, arabinoxylan.

In one embodiment, the substrate is an activated furanoside donor selected from the list comprising p-nitrophenyl α-L-arabinofuranoside, dinitrophenyl α-L-arabinofuranoside, chloronitrophenyl α-L-arabinofuranoside, 1-thioimidoyl α-L-arabinofuranose, 5-bromo-indolyl α-L-arabinofuranoside, p-nitrophenyl β-D-galactofuranoside, dinitrophenyl β-D-galactofuranoside, chloronitrophenyl β-D-galactofuranoside, 1-thioimidoyl β-D-galactofuranose, p-nitrophenyl 6-deoxy-6-fluoro-β-D-galactofuranoside, dinitrophenyl 6-deoxy-6-fluoro-β-D-galactofuranoside, chloronitrophenyl 6-deoxy-6-fluoro-β-D-galactofuranoside, 1-thioimidoyl 6-deoxy-6-fluoro-β-D-galactofuranose, 5-bromo-indolyl β-D-galactofuranoside, p-nitrophenyl β-D-fucofuranoside, 5-bromo-indolyl β-D-fucofuranoside and mixtures thereof (Chlubnova et al, Org. Biomol. Chem. 2010, 8, 2092-2102; Tanaka et al, Chem. Commun. 2008, 2016-2018)

In one embodiment of the invention, the product of interest is a furanoside, preferably an alkyl-arabinofuranoside or an alkenyl-furanoside.

One advantage of the invention is that the process of the invention does not lead to any mixture or by-product, and result in the direct synthesis of the furanosides of interest. Especially, no accumulation of by-products, resulting from the auto-condensation or transglycosylation of the substrate, was observed.

In one embodiment of the invention, the alcohol acceptor is an aliphatic alcohol, preferably selected from the group comprising methanol, ethanol, propanol, isopropanol, butanol, pentanol and hexanol. In another embodiment of the invention, the alcohol acceptor is solketal. In another embodiment of the invention, the alcohol acceptor is an allylic alcohol. In another embodiment of the invention, the alcohol acceptor is an alkenic alcohol.

The present invention also relates to a process for producing alkyl furanosides from polysaccharide raw materials, comprising contacting said polysaccharide raw materials with an enzyme, preferably a native or mutant Araf51 enzyme, in presence of an alcohol acceptor.

In one embodiment of the invention, arabinan is contacted with an Araf51 enzyme in presence of methanol to produce methyl-α-L-arabinofuranoside—

Examples of resulting alkyl furanosides include, but are not limited to methyl-furanoside, ethyl-furanoside, propyl-furanoside, butyl furanoside, pentyl-furanoside, hexyl-furanoside, heptyl-furanoside, octyl-furanoside, arabinofuranosides, polyfuranosides.

According to an embodiment, resulting alkyl furanosides of interest include methyl-α-L-arabinofuranoside, ethyl-α-L-arabinofuranoside, propyl-α-L-arabinofuranoside, i-propyl-α-L-arabinofuranoside, n-butyl-α-L-arabinofuranoside, n-pentyl-α-L-arabinofuranoside, n-hexyl-α-L-arabinofuranoside.

According to an embodiment, the product of interest is an alkyl-furanoside, preferably an alkyl-arabinofuranoside, more preferably the product is selected from the group comprising butyl furanoside, n-butylfuranoside, polyfuranoside, octyl-furanoside, methyl α-L-arabinofuranoside; or an alkenyl-furanoside or an allylic furanoside.

### [Mutant Enzyme]

Another object of the invention is a mutant Araf51 enzyme showing improved transglycosylation activity in comparison with the native wild-type (wt) Araf51 enzyme.

In one embodiment, the mutant Araf51 enzyme is encoded by a nucleotide sequence selected from the group consisting of SEQ ID NO: 2, SEQ ID NO: 3, SEQ ID NO: 4, SEQ ID NO: 5 and SEQ ID NO: 6.

In one embodiment, the mutant Araf51 enzyme may act using glycosyl donors selected from the list comprising natural polysaccharides from lignocellulosic biomass, natural arabinan polymers, arabinoxylan polymers, pentoses from hemicellulose, p-nitrophenyl α-L-arabinofuranoside, dinitrophenyl α-L-arabinofuranoside, chloronitrophenyl α-L-arabinofuranoside, 1-thioimidoyl α-L-arabinofuranose, 5-bromo-indolyl α-L-arabinofuranoside, p-nitrophenyl β-D-galactofuranoside, dinitrophenyl β-D-galactofuranoside, chloronitrophenyl β-D-galactofuranoside, 1-thioimidoyl β-D-galactofuranose, p-nitrophenyl 6-deoxy-6-fluoro-β-D-galactofuranoside, dinitrophenyl 6-deoxy-6-fluoro-β-D-galactofuranoside, chloronitrophenyl 6-deoxy-6-fluoro-β-D-galactofuranoside, 1-thioimidoyl 6-deoxy-6-fluoro-β-D-galactofuranose, 5-bromo-indolyl β-D-galactofuranoside, p-nitrophenyl β-D-fucofuranoside, 5-bromo-indolyl β-D-fucofuranoside and mixtures thereof (Chlubnová et al, Org. Biomol. Chem. 2010, 8, 2092-2102; Tanaka et al, Chem. Commun. 2008, 2016-2018).

In one embodiment, the mutant Araf51 enzyme may act using alcohol acceptors selected from the list comprising aliphatic alcohols, such as, for example, methanol, ethanol, propanol (such as, for example n-propanol), isopropanol, butanol (such as, for example, n-butanol), pentanol (such as, for example, n-pentanol), hexanol (such as, for example, n-hexanol), solketal, allylic alcohols or alkenic alcohols.

In one embodiment, the mutant Araf51 enzyme is not inhibited in presence of alcohol acceptors.

In one embodiment, the mutant Araf51 enzyme of the invention does not catalyze the auto-condensation of the glycosyl donor in the presence of an alcohol acceptor.

In one embodiment of the invention, the mutant Araf51 enzyme presents an increased kinetic conversion rate. In one embodiment, when measuring the conversion rate of the mutant Araf51 enzyme, the curve reaches a plateau in less than or equal to about 140 minutes, preferably less than or equal to about 120, 100, 80, 60, 40 minutes, more preferably in less than or equal to about 20 minutes.

In one embodiment, said mutant Araf51 enzyme presents a molar conversion yield of more than 30%, preferably of more than 50%, more preferably of more than 70%, even more preferably of more than 90%.

In one embodiment, the mutant Araf51 enzyme uses n-butanol, as alcohol acceptor. In one embodiment, the mutant Araf51 enzyme present a transglycosylation conversion rate of more than 80%, preferably more than 90%, even more preferably of about 92%.

In one embodiment, the transglycosylation conversion is carried out in less than 40 minutes, preferably less than 30 minutes, more preferably in about 20 minutes.

In one embodiment, the mutant Araf51 enzyme uses n-propanol, as alcohol acceptor. In one embodiment, the mutant Araf51 enzyme present a transglycosylation conversion rate of more than 80%, preferably more than 90%, even more preferably of about 96%. In one embodiment, the transglycosylation conversion is carried out in less than 100 minutes, preferably less than 80 minutes, more preferably in about 60 minutes.

In one embodiment, the mutant Araf51 enzyme uses isopropanol as alcohol acceptor. In one embodiment, the mutant Araf51 enzyme present a transglycosylation conversion rate of more than 20%, preferably more than 30%, even more preferably of about 38%. In one embodiment, the transglycosylation conversion is carried out in less than 100 minutes, preferably less than 80 minutes, more preferably in about 60 minutes.

In one embodiment, the mutant Araf51 enzyme uses n-pentanol, as alcohol acceptor.

In one embodiment, the mutant Araf51 enzyme uses n-hexanol, as alcohol acceptor. In one embodiment, the mutant Araf51 enzyme present a transglycosylation conversion rate of more than 80%, preferably more than 90%, even more preferably of about 94%. In one embodiment, the transglycosylation conversion is carried out in less than 140 minutes, preferably less than 130 minutes, more preferably in about 120 minutes.

In one embodiment, the mutant Araf51 enzyme is selected from the group comprising proteins encoded by the nucleotide sequence SEQ ID NO: 2, SEQ ID NO: 3, SEQ ID NO: 4, SEQ ID NO: 5 and SEQ ID NO: 6.

### [Screening Method]

Another object of this invention is a screening method for identifying mutant Araf51 enzyme showing improved activity of transglycosylation of a selection substrate in comparison with the native wild-type (wt) Araf51 enzyme. In one embodiment, said selection substrate is pNP-Araf (p-nitrophenyl α-L-arabinofuranoside).

In one embodiment, said mutant Araf51 enzyme is obtained by mutagenesis, such as, for example, random mutagenesis or targeted mutagenesis. Method that may be used for inducing mutagenesis are well-known for the person skilled in the art, and include, without limitation, PCR based method. An example of random mutagenesis experiment is described in the Examples. In one embodiment, the selection of hydrolytic mutants, i.e. enzymes able to recognize the arabinofuranosyl substrate and to remove the aglycone part for further hydrolysis and/or transglycosylation reactions was performed thanks to a chromogenic substrate.

In one embodiment, the following protocol may be used for comparing transglycosylation (in presence of the alcohol acceptor) and hydrolytic activities (in absence of the alcohol) of a mutant Araf51 enzyme using as selection substrate pNP-Araf:

Mutants and Araf51 WT enzymes were incubated at the same final concentration with or without alcohol acceptor. The release of para-nitrophenol was measured at 405 nm during 5 min using a spectrophotometer, such as, for example, a Microplate Spectrophotometer Powerwase XS/XS2 (Biotek). The initial activities of the enzyme and mutated enzymes were determined using the UV curve of the enzymatic assays. This enabled to compare the slope between Araf51 WT and the one of the mutants with or without the alcohol acceptors, and highlighted the mutants of interest. The mutated enzymes presenting a higher slope than the one of the Araf51 WT, in presence of alcohol, showing higher reaction activations (meaning that transglycosylation was preferred) correspond to enzyme of the invention.

A detailed protocol for screening mutant Araf51 enzyme of the invention is shown in the Examples.

### Industrial Application and Advantages

The innovative approach developed in this invention consists in using plant raw material, such as, for example, furanosyl-containing polysaccharides, which is still hardly exploited, for the preparation of a large family of glycosides.

This green and sustainable methodology is based on the use of wild-type and randomly mutated enzymes as biocatalysts, obtained from well-known molecular biological techniques.

Indeed, while sugars and especially pentoses are available from the hydrolysis of wheat or corn co-products, the manufacture of pentoses remains problematic. For instance, their extraction from biomass usually requires elevated temperatures and pressures increasing the overall cost of the process.

The methodology herein described for the direct conversion of natural arabinans and arabinoxylans into arabinosides allows a diminution of the chemicals required (no acid or base agents are needed). Moreover it could be applied to the preparation of a large variety of compounds depending on the nature of the alcohol acceptors.

The main purposes may consist in the synthesis of chemicals as valuable building blocks and/or molecules of interest:
From n-butanol as alcohol, n-butylfuranoside and polyfuranosides could be obtained, as new non-ionic surfactants likely to be included in the APGs family. Moreover butyl-based APGs are used as hydrotropes in detergent industry and as foam boosters in personal care products.
From allylic or alkenic alcohol acceptors, alkenyl-furanosides could be accessed and likely to be polymerized to get furanoside-containing polymers from renewable source. The resulting biodegradable and low-cost natural materials, nowadays commonly called “biocomposite” are hardly requested by the plastic industry in order to reduce the environmental pollution resulting from non-biodegradable plastic waste.

### BRIEF DESCRIPTION OF THE DRAWINGS

FIG. 1 is a combination of graphs showing the measurement of p-nitrophenol during enzymatic reaction with pNP-Araf as donor with n-butanol (▪) or without (x). (A) pNPOH release from wt enzyme. (B) pNPOH release from M12 mutant.

FIG. 2 is a combination of graphs showing the kinetic conversion of a) the enzyme-catalyzed transglycosylation with pNP-Araf as donor and butanol as acceptor (x), b) the enzyme-catalyzed consumption of pNP-Araf (♦).

FIG. 3 is a graph showing the measurement of arabinose released during hydrolytic reaction starting from branched arabinan (▪), debranched arabinan (x) and arabinoxylan (Δ) by Araf51 WT.

### EXAMPLES

The present invention is further illustrated by the following examples.

### Example 1

### Materials and Methods

### Expression and Purification of Araf51 and Mutant Derivatives

Plasmid pET28a (Novagen) contains Araf51 wild type and kanamycin resistance genes. Plasmid pCR®2.1-TOPO® (3.9 kb) contains encoding mutated enzymes genes as well as ampicillin and kanamycin resistance genes. These plasmids were under the control of T7 promoter. The enzymes were produced in Escherichia coli BL21 DE3 cells cultured in LB (Luria Bertani) broth containing 0.1 mM of the corresponding selective agent at 37° C. The cells were grown to mid-exponential phase [Absorbance, A₅₅₀: 0.7] at which point isopropyl-β-D-thiogalactopyranoside was added to a final concentration of 1.0 mM and the cultures were incubated for 14 h at 37° C. After centrifugation (20 min at 4000 rpm) and sonication (3×10 s), the supernatant was heated at 70° C. for 15 min to remove a major amount of thermolabile proteins and centrifuged again at 20000 rpm for 20 min. Protein concentrations were determined by the Bradford method.

### Random Mutagenesis

Random mutagenesis was performed by GeneMorph II Random Mutagenesis kit (Stratagene) using mutagenic PCR. The open reading frame encoding Araf51 was amplified using the primers: forward T7 Promoter TACGACTCACTATAGGGGAA (SEQ ID NO: 7) and reverse T7 Promoter GTGAGTCGTATTAATTTCGCGGT (SEQ ID NO: 8) (250 ng/μL of each primer). For low mutations rates (mutation frequency 0-4.5 mutations/kb), 500 ng of the initial target DNA were mixed with 250 ng of each primer, 1 μL of 40 mM dNTP mix (final concentration of 200 μM each), 5 μL of 10× Mutazyme II reaction buffer and 1 μL of Mutazyme II DNA polymerase (2.5 U/μL) completed to 50 μL with H₂O. The reaction was thermocycled as follows: one hot start cycle (95° C., 2 min) then 10 cycles: first the denaturing step (95° C., 30 s), the hybridation step (60° C., 30 s) and the elongation step performed for 1 min/kb (72° C., 7 min); and finally one cycle at 72° C. for 10 min.

Mutagenesis PCR products were directly cloned into a plasmid vector using the TOPO TA Cloning® Invitrogen protocol. The fresh PCR product (2 μL) was mixed with the different reagents provided in the TOPO TA Cloning® Invitrogen kit: 1 μL salt solution, 1 μL pCR®2.1-TOPO® vector and H₂O was added up to a final volume of 6 μL. The reaction was incubated for 5 min at room temperature (22-23° C.).

Chemical Transformation in E. coli Top 10 One Shot®

2 μL of the TOPO® cloning reaction was added to a vial of E. coli Top 10 One Shot® Chemically Competent (Invitrogen) and incubated on ice for 15 min. Heat-shock was realized at 42° C. during 30 s and the tubes were immediately transferred to ice. The cells were incubated with S.O.C. medium for 1 h at 37° C. following by incubation in LB liquid media supplemented by 0.1 mM of ampicillin to prevent pET28a plasmid containing E. coli to grow. Extraction by Strataprep Plasmid Mininprep kit was performed to allow another transformation of the plasmid pCR®2.1-TOPO® into E. coli BL21 DE3.

Transformed cells (300 μL) were spread on nitrocellulose membrane placed on LB agar supplemented with 0.1 mM of kanamycin and were grown at 37° C. overnight. The nitrocellulose membrane was transferred onto another plate with 0.1 mM kanamycin LB media, IPTG and 5-bromo-indolyl-α-L-arabinofuranoside (1.5 mM) and incubated overnight (37° C.).

### Screening of Mutants

The blue colonies were selected and cultivated in 5 mL 0.1 mM kanamycin LB media. The enzymes were purified as previously described in this section and analyzed for their transglycosylation activities. As previously, protein concentration was estimated by Bradford method.

Enzyme assays were performed to compare transglycosylation activities (presence of the alcohol acceptor) to hydrolytic activities (absence of the alcohol). Mutants and Araf51 WT enzymes were incubated in pH 8 Tris HCl 50 mM buffer with 20 mM pNP-Araf, 20% (v/v) DMSO, with or without 25% (v/v) alcohol Qsp 140 μL at 50° C. The final concentration in enzyme will reach 0.017 mg/mL, the collected volume having to be adapted to each attempt following the determination of the initial concentration by the Bradford method. Furthermore, each enzymatic extract was diluted to a final concentration of 0.017 mg/mL. The release of para-nitrophenol was measured at 405 nm during 5 min (Microplate Spectrophotometer Powerwase XS/XS2, Biotek) and data evaluated with GenS Data Analysis Software (Biotek). The initial activities of the enzyme and mutated enzymes were determined using the UV curve of the enzymatic assays. This enabled to compare the slope between Araf51 WT and the one of the mutants with or without the alcohol acceptors, and highlighted the mutants of interest. The mutated enzymes presenting a higher slope than the one of the Araf51 WT, in presence of alcohol, showed higher reaction activations, meaning that transglycosylation was preferred.

Transglycosylation Reactions with Mutated Enzymes

Enzymatic reactions were run from 20 mM pNP-Araf (4.3 mg) and 200 μL of alcohol acceptor incubated in pH 8 Tris HCl 50 mM buffer with 160 μL of DMSO, in the presence of the enzyme (a final concentration of 0.017 mg/mL is required), and finally completed to a final volume of 800 μL and maintained at 50° C. during 3 h. Aliquots (100 μL) of the enzymatic reaction mixture were withdrawn at several times and directly freezed with liquid nitrogen. After complete lyophilization, samples were solubilized in 500 μL of MeOD to enable the analysis by NMR.

Transglycosylation activities using pNP-Araf as glycosyl donor were determined by ¹H NMR. By following the decrease of the pNP-Araf signal (aromatic proton δ=8.21 ppm and/or anomeric proton δ=5.66 ppm) and the release of p-nitrophenol's signal (aromatic proton δ=8.12 ppm) corresponding to the hydrolysis and the transglycosylation of the donor, the residual starting material can easily be quantified. The transglycosylation products were visualized by the apparition of the anomeric proton signal of the furanoside and/or the signal of the alkyl group protons. By reporting the relation between the protons signals, the resulting conversion rates were evaluated.

### Methyl-α-L-arabinofuranoside (15.9 mg, 88%)

This reaction was performed from 30 mg of pNP-Araf and in the presence of the wt Araf51.

¹H NMR (400 MHz, CD₃OD): δ=4.75 (d, J_{1,2}=1.6 Hz, 1H, H-1), 3.93 (dd, J_{2,3}=3.6 Hz, 1H, H-2), 3.90 (m, 1H, H-4), 3.82 (dd, J_{3,4}=6.4 Hz, 1H, H-3), 3.74 (dd, J_{4,5a}=3.2 Hz, 1H, H-5a), 3.64 (dd, J_{4,5b}=4.4 Hz, J_{5a,5b}=11.6 Hz, 1H, H-5b), 3.36 (s, 3H, CH₃) ppm. ¹³C NMR (100 MHz, CD₃OD): δ=110.5 (C-1), 85.5 (C-4), 83.3 (C-2), 78.6 (C-3), 63.0 (C-5), 55.3 (CH₃).

### Ethyl-α-L-arabinofuranoside (15.4 mg, 78%)

This reaction was performed from 30 mg of pNP-Araf and in the presence of the wt Araf51.

¹H NMR (400 MHz, CD₃OD): δ=4.86 (s, 1H, H-1), 3.94 (dd, J_{1,2}=2 Hz, J_{2,3}=4 Hz, 1H, H-2), 3.91 (m, J_{3,4}=6.8 Hz, J_{4,5}=5.2 Hz, 1H, H-4), 3.82 (dd, 1H, H-3), 3.75 (m, 2H, H-5a, CH₂-a CH₃), 3.625 (dd, J_{5a,5b}=12 Hz, 1H, H-5b), 3.50 (dq, J_{CH2a,CH2b}=9.6 Hz, J_{CH2,CH3}=7.2 Hz, 1H, CH₂-b-CH₃), 3.31 (t, 3H, CH₂CH₃) ppm. ¹³C NMR (100 MHz, CD₃OD): δ=109.2 (C-1), 85.2 (C-4), 83.6 (C-2), 78.6 (C-3), 64.2 (CH₂CH₃), 63.0 (C-5), 15.4 (CH₂CH₃).

### Propyl-α-L-arabinofuranoside (15.8 mg, 74%)

This reaction was performed from 30 mg of pNP-Araf and in the presence of the M20 mutant.

¹H NMR (400 MHz, CD₃OD): δ=4.85 (d, J_{1,2}=2 Hz, 1H, H-1), 3.95 (dd, J_{2,3}=4 Hz, 1H, H-2), 3.91 (m, 1H, H-4), 3.82 (dd, J_{3,4}=6 Hz, 1H, H-3), 3.74 (dd, J_{4,5a}=3.2 Hz, J_{5a,5b}=12 Hz, 1H, H-5a), 3.66 (m, 3H, H-5b, CH₂CH₂CH₃), 1.67 (dd, J_{CH2,CH2}=6.8 Hz, J_{CH2,CH2}=1314 Hz, 1H, CH₂CH₂CH₃), 0.94 (t, J_{CH3,CH2}=7.2 Hz, 3H, CH₂CH₂CH₃) ppm. ¹³C NMR (100 MHz, CD₃OD): δ=109.6 (C-1), 85.0 (C-4), 83.4 (C-2), 78.6 (C-3), 68.4 (CH₂CH₂CH₃), 63.0 (C-5), 25.4 (CH₂CH₂CH₃), 15.2 (CH₂CH₂CH₃).

i-Propyl-α-L-arabinofuranoside (12.6 mg, 60%)

This reaction was performed from 30 mg of pNP-Araf and in the presence of the M22 mutant.

¹H NMR (400 MHz, CD₃OD): δ=4.96 (d, J_{1,2}=1.6 Hz, 1H, H-1), 3.91 (m, 3H, H-2, H-4, CH(CH₃)₂), 3.87 (dd, J_{2,3}=4 Hz, J_{3,4}=6.4 Hz, 1H, H-3), 3.74 (dd, J_{4,5a}=2.8 Hz, J_{5a,5b}=12 Hz, 1H, H-5a), 3.62 (dd, J_{4,5b}=5.2 Hz, 1H, H-5b), 1.2 (d, J_{CH3,CH}=6 Hz, 3H, CH(CH₃)₂), 1.57 (d, 3H, CH(CH₃)₂) ppm. ¹³C NMR (100 MHz, CD₃OD): δ=107.6 (C-1), 84.9 (C-4), 83.8 (C-2), 78.6 (C-3), 64.2 (CH(CH₃)₂), 63.0 (C-5), 23.9 (CH(CH₃)₂), 21.9 (CH(CH₃)₂).

n-Butyl-α-L-arabinofuranoside (15.4 mg, 68%)

This reaction was performed from 30 mg of pNP-Araf and in the presence of the M12 mutant.

¹H NMR (400 MHz, CD₃OD): δ=4.84 (d, J_{1,2}=2 Hz, 1H, H-1), 3.94 (dd, J_{2,3}=4 Hz, 1H, H-2), 3.90 (m, 1H, H-4), 3.82 (dd, J_{3,4}=6.8 Hz, 3H, H-3), 3.73 (m, 2H, H-5b, CH₂CH₂CH₂CH₃), 3.62 (dd, J_{4,5a}=5.2 Hz, J_{5a,5b}=12 Hz, 1H, H-5a), 3.42 (dd, J_{CH2,CH2}=6.4 Hz, J_{CH2,CH2}=13.6 Hz, 1H, CH₂CH₂CH₂CH₃), 1.57 (dt, J_{CH2,CH2}=3.2 Hz, J_{CH2,CH2}=15.2 Hz, 2H, CH₂CH₂CH₂CH₃), 1.40 (td, 2H, CH₂CH₂CH₂CH₃), 0.937 (t, J_{CH2,CH3}=7.2 Hz, 3H, CH₂CH₂CH₂CH₃) ppm. ¹³C NMR (100 MHz, CD₃OD): δ=109.4 (C-1), 85.1 (C-4), 83.6 (C-2), 78.7 (C-3), 68.5 (CH₂CH₂CH₂CH₃), 63.0 (C-5), 32.8 (CH₂CH₂CH₂CH₃), 20.36 (CH₂CH₂CH₂CH₃), 14.2 (CH₂CH₂CH₂CH₃).

n-Pentyl-α-L-arabinofuranoside (16.1 mg, 66%)

This reaction was performed from 30 mg of pNP-Araf and in the presence of the M60 mutant.

¹H NMR (400 MHz, CD₃OD): δ=4.84 (d, J_{1,2}=2 Hz, 1H, H-1), 3.94 (dd, J_{2,3}=4 Hz, 1H, H-2), 3.90 (m, 1H, H-4), 3.82 (dd, J_{3,4}=6.8 Hz, 1H, H-3), 3.73 (dd, J_{4,5}=3.2 Hz, J_{5a,5b}=12 Hz, 1H, H-5a), 3.71 (dt, J_{CH2,CH2}=6.4 Hz, J_{H,H}=10 Hz, J_{CH2,CH2}=3.2 Hz, 1H, CH₂CH₂CH₂CH₂CH₃), 3.62 (dd, J_{4,5b}=5.2 Hz, 1H, H-5b), 3.88 (dt, J_{CH2,CH2}=2.8 Hz, 1H, CH₂CH₂CH₂CH₂CH₃), 1.59 (dt, J_{CH2,CH2}=7.2 Hz, J_{CH2,CH2}=6.8 Hz, 2H, CH₂CH₂CH₂CH₂CH₃), 1.35 (m, 4H, CH₂CH₂CH₂CH₂CH₃), 0.92 (t, J_{CH2,CH3}=7.2 Hz, 3H, CH₂CH₂CH₂CH₂CH₃) ppm. ¹³C NMR (100 MHz, CD₃OD): δ=109.4 (C-1), 85.1 (C-4), 83.6 (C-2), 78.7 (C-3), 68.8 (CH₂CH₂CH₂CH₂CH₃), 63.0 (C-5), 30.4 (CH₂CH₂CH₂CH₂CH₃), 29.5 (CH₂CH₂CH₂CH₂CH₃), 23.5 (CH₂CH₂CH₂CH₂CH₃), 14.4 (CH₂CH₂CH₂CH₂CH₃).

n-Hexyl-α-L-arabinofuranoside (18.5 mg, 71%)

This reaction was performed from 30 mg of pNP-Araf and in the presence of the M57 mutant.

¹H NMR (400 MHz, CD₃OD): δ=4.84 (d, 0.42=2 Hz, 1H, H-1), 3.94 (dd, J_{2,3}=4 Hz, 1H, H-2), 3.91 (m, 1H, H-4), 3.82 (dd, J_{3,4}=6.4 Hz, 1H, H-3), 3.74 (dd, J_{4,5}=3.2 Hz, J_{5a,5b}=12 Hz, 1H, H-5a), 3.71 (dt, J_{CH2,CH2}=6.8 Hz, J_{H,H}=9.6 Hz, J_{CH2,CH2}=2.8 Hz, 1H, CH₂CH₂CH₂CH₂CH₂CH₃), 3.62 (dd, J_{4,5b}=5.6 Hz, 1H, H-5b), 3.41 (dt, J_{CH2,CH2}=6.4 Hz, J_{CH2,CH2}=3.2 Hz, 1H, CH₂CH₂CH₂CH₂CH₂CH₃), 1.59 (dt, J_{CH2,CH2}=7.6 Hz, 2H, CH₂CH₂CH₂CH₂CH₂CH₃), 1.33 (m, 6H, CH₂CH₂CH₂CH₂CH₂CH₃), 0.91 (t, J_{CH2,CH3}=6.8 Hz, 3H, CH₂CH₂CH₂CH₂CH₂CH₃) ppm. ¹³C NMR (100 MHz, CD₃OD): δ=109.4 (C-1), 85.1 (C-4), 83.6 (C-2), 78.7 (C-3), 68.8 (CH₂CH₂CH₂CH₂CH₂CH₃), 63.0 (C-5), 32.8 (CH₂CH₂CH₂CH₂CH₂CH₃), 30.7 (CH₂CH₂CH₂CH₂CH₂CH₃), 26.9 (CH₂CH₂CH₂CH₂CH₂CH₃), 23.7 (CH₂CH₂CH₂CH₂CH₂CH₃), 14.4 (CH₂CH₂CH₂CH₂CH₂CH₃).

Transglycosylation Reactions with Arabinan as a Donor

Enzymatic assays were carried out using arabinan as a donor substrate (88% pure from Megazyme). 5 mL reaction solution was prepared to a final concentration of 30 mg/mL of arabinan containing 20% of methanol in a 50 mM Tris HCl buffer (pH 8). The reaction was incubated with the WT Araf51 (0.2 mg/mL) at 50° C. during 72 h. Reaction mixture was lyophilized and the residue was purified by column chromatrography on silica gel (9:1 CH₂Cl₂-MeOH) to give a colorless oil corresponding to the transglycosylation product, methyl α-L-arabinofuranoside, in 15% yield (22 mg).

### Results and Discussion

### Identification of Improved Mutants of Araf51 for Transglycosylation.

We first performed a random mutagenesis of the Araf51 WT gene by error prone PCR and allowing the access to PCR libraries of Araf51 mutants. They were then screened in a two steps procedure. The extracted plasmid DNA library form mutagenesis was transformed in Escherichia coli BL21 strain. This resulted in colonies that grew on LB plates containing the 5-bromo-indolyl α-L-arabinofuranoside, “X-Araf” (1.5 mM), a chromogenic arabinofuranoside substrate¹⁰ likely to be transported through the E. coli membranes. First step consisted in the selection of the overexpressed mutated enzymes based on their ability to use “X-Araf” as a donor of an arabinofuranosyl entity. Therefore the hydrolytic activities of the enzymes were revealed by the appearance on agar plates of the blue color due to the resulting air-oxidized di-indolyl compound (scheme 2).

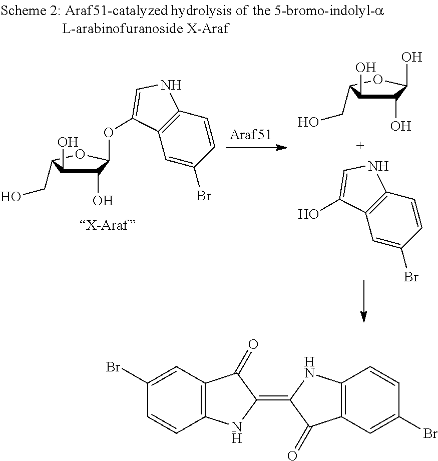

During the second step, the selected mutants were isolated and the corresponding enzyme extracts were produced to evaluate their ability to catalyze the transglycosylation of p-nitrophenyl α-L-arabinofuranoside pNP-Araf as a donor and various aliphatic alcohols as acceptors (scheme 3). A panel of 90 blue colonies was withdrawn for kinetic reaction analysis and each enzyme (0.017 mg/mL) was tested with 20 mM pNP-Araf with or without alcohol (25% v/v) as an acceptor at 50° C. in 50 mM Tris HCl buffer (pH 8). Aliphatic alcohols with increasing chain length, from methanol to hexanol, were tested, as well as solketal.

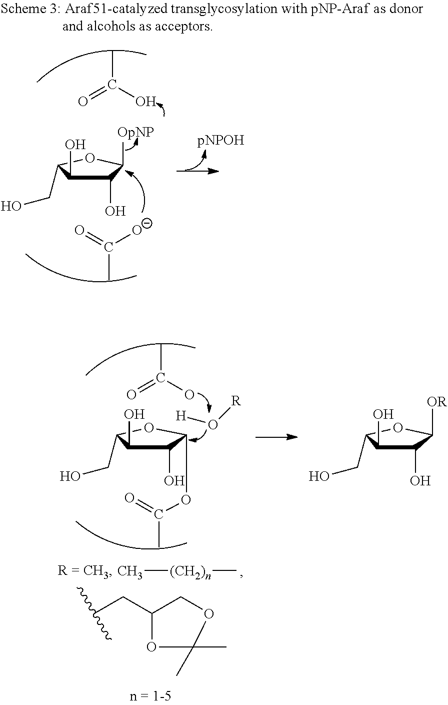

In presence of activated donor, the enzyme followed the Michealis-Menten model. The reactions were analyzed during the initial reaction time where the deglycosylation of the glycosyl-enzyme intermediate leading to the formation of the product was the rate-determining step of the reaction. The action of the pNP-Araf as donor was confirmed by the released of p-nitrophenol (pNPOH), monitoring by spectrometric analysis (X=405 nm). Upon addition of an acceptor, two separate reactions entered in competition: hydrolysis and transglycosylation. The first one occurred when the glycosyl-enzyme intermediate accepts a molecule of water and the last one, when the alcohol is used as the nucleophile. In presence of the suitable alcohol, the transglycosidase mutants exhibited an improved activity. The turn-over was increased, associated with an increase of the p-nitrophenol released (enhanced glycosylation). In the opposite case, the wild-type enzyme or the mutant could be inhibited in presence of alcohol acceptors.

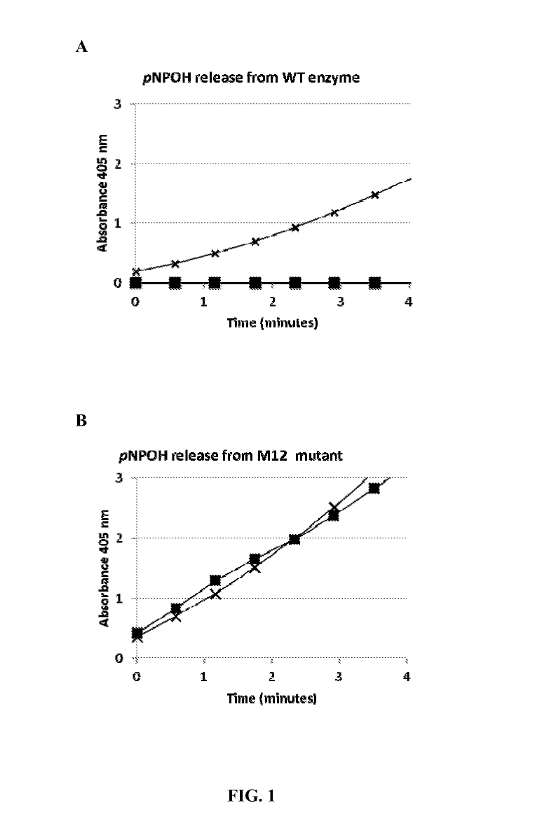

In this example (FIG. 1), the release of p-nitrophenol was monitored during the WT Araf51-catalyzed reaction with pNP-Araf with (▪) or without the presence of n-butanol (x). The kinetic curve from Araf51 WT presented a slight slope reduction after addition of acceptor due to inactivation. In comparison, pNPOH release with the selected transglycosidase mutant M12 exhibited a significant increase in presence of n-butanol.

To validate this screening strategy, the activities of the best mutants thus screened in the presence of these alcohols were quantitatively analyzed by NMR spectroscopy to confirm the results and to determinate the conversion rate of the furanosyl substrate into the alkyl furanosides. The transglycosylation reaction medium was withdrawn at several times and each sample was analyzed by ¹H NMR experiment. Proton's signals belonging to the product and others from the starting material were identified and their integration allowed the evaluation of the conversion rate.

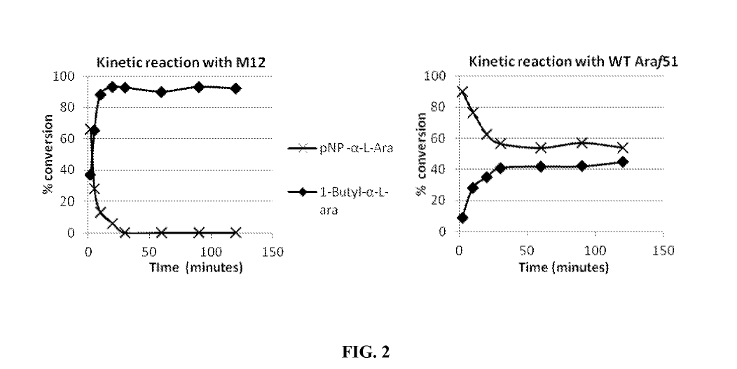

In the previous example based on the n-butanol, we observed a significant increase in the transglycosylation activity. The maximal conversion calculated as the molar yield of pNP-Araf transferred to the acceptor reached 40% with the WT enzyme and more than 95% with the M12 mutant (FIG. 2). With the WT Araf51, 50 min were necessary to convert half of the initial starting material pNP-Araf (20 mM) in the butyl furanoside while the donor was completely consumed after only 20 min with the M12 mutant. It is worth mentioning that in these described conditions of reaction, the selected mutants are able to catalyze the transglycosylation reaction on an aliphatic alcohol rather than the self-condensation one. Moreover, the hydrolysis of the arabinofuranoside products is not observed, whatever the nature of the biocatalyst. Finally, no transglycosylation by-products such as the β-anomer of the furanosides were formed, confirming the diastereoselectivity of the mutants according to the glycosidic bond.

The screening underlined 5 interesting clones, each one corresponding to a different acceptor (n-propanol, isopropanol, n-butanol, n-pentanol and n-hexanol) (Table 1). Compared to the performance of the WT Araf51, they were all more efficient in the transglycosylation reaction using pNP-Araf as the glycosyl donor allowing the syntheses of various alkyl arabinofuranosides with good to excellent conversion yields.

TABLE 1
Comparison of the rates and the conversion yields for the
transglycosylation reactions mediated by WT
Araf51 and the selected mutated enzymes.

| | Mutants | | Araf51 WT | |
|---|---|---|---|---|
| Alcohol | Transglycosylation | Time | transglycosylation | Time |
| acceptor | conversion | (min) | conversion | (min) |
| 1-butanol | 92% (M12) | 20 | 42% | 50 |
| 1-propanol | 96% (M20) | 60 | 85% | 140 |
| isopropanol | 38% (M22) | 60 | 30% | 90 |
| 1-pentanol | 96% (M60) | 120 | 72% | 120 |
| 1-hexanol | 94% (M57) | 120 | 37% | 120 |

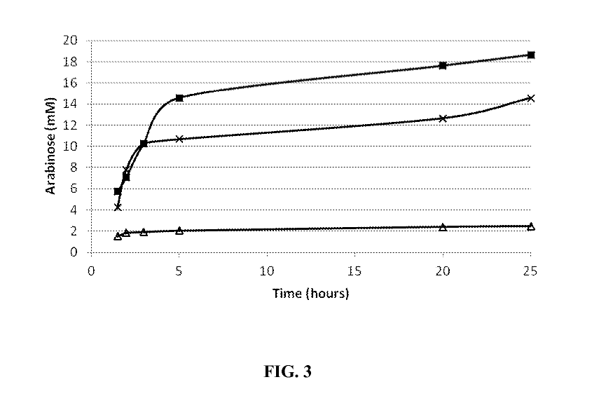

Subsequently, branched and linear arabinans were also evaluated as a potential source of arabinose for the synthesis of alkyl arabinofuranosides using the herein developed biotechnological strategy. Three types of natural polymers (branched arabinan, debranched arabinan and arabinoxylan) could likely to be used as substrate donors. FIG. 3 is related to the evolution of the arabinose released monitoring by HPLC analysis (light scattering detection) from these different sources of arabinan and demonstrated that branched sugar beet arabinan was preferably hydrolysed by the WT Araf51. This is in accordance with the enzyme specificity for α-1,3- and α-1,5-linked arabinofuranose residues.

Initial enzymatic assays were performed using 30 mg/mL sugar beet arabinan (88% pure from Megazyme) and methanol (25% v/v) as alcohol acceptor. In presence of the wild type Araf51, the reaction was incubated at 50° C. in 50 mM Tris HCl buffer (pH 8) during 72 h. After purification, we specifically obtained the target product, 22 mg of methyl α-L-arabinofuranoside, that corresponds to a 15% yield, keeping in mind that this last one is composed of Ara: Gal: Rha: GalUA (88:3:2:7).

Experiments were run in the same conditions (50° C. in 50 mM Tris HCl buffer (pH 8) during 72 h) with the selected mutated enzyme. Roughly it appeared that some of them, especially M12 and M20 enzymes were able to catalyze transglycosylation reactions from branched arabinan (5 mg/mL) with different alcohols as acceptors to afford alkyl arabinofuranosides, especially propyl arabinofuranoside, in a concentration up to 2-fold the one obtained with the wild-type.

This first result is a good start to develop our project to obtain alkyl furanosides with eco-friendly syntheses, optimization of reaction parameters have to be studied to induce the highest transglycosylation capacity of the Araf51 from natural polymers such as arabinans and arabinoxylans.

1. A process for enzymatically converting a furanosyl-containing polysaccharide substrate in a product of interest which is a furanoside, said process comprising contacting said substrate with an enzyme in presence of an alcohol acceptor.

2. The process of claim 1 being a one step process.

3. The process of claim 1, wherein the enzymatic conversion is a transglycosylation.

4. The process of claim 1, wherein the enzyme is selected from the group comprising proteins of the GH51 family, such as, for example, Araf51 GH51 from Clostridium thermocellum (encoded by the nucleotide sequence SEQ ID NO: 1), Tm-AFase GH51 from Thermotoga maritima(SEQ ID NO: 9), AbfD3 GH51 from Thermobacillus xylaniliticus (SEQ ID NO: 10), AbfAT-6 GH51 from Geobacillus stearothermophilus (SEQ ID NO: 11), AbfA GH51 from Aspergillus oryzae (SEQ ID NO: 12); GH 43 from Bacillus subtilis (SEQ ID NO: 13); Abf51A from Cellvibrio japonicus (SEQ ID NO: 14); CBM42 GH42 from Streptomyces avermitilis (SEQ ID NO: 15); AkabfB GH54 Aspergillus kawachii (SEQ ID NO: 16); and α-ara pI from Aspergillus terreus (SEQ ID NO: 17).

5. The process of claim 1, wherein the enzyme is an Araf51 enzyme from Clostridium thermocellum (SEQ ID NO: 1).

6. The process of claim 1, wherein the enzyme is a native Araf51 enzyme.

7. The process of claim 1, wherein the enzyme is a mutant Araf51 enzyme, wherein said mutant enzyme presents at least one of the following features:
no inhibition in presence of alcohol acceptors;
increased kinetic conversion rate; and/or
molar conversion yield of more than 30%.

8. The process of claim 1, wherein the enzyme is a mutant Araf51 enzyme encoded by a nucleotide sequence selected from the group consisting of SEQ ID NO: 2, SEQ ID NO: 3, SEQ ID NO: 4, SEQ ID NO: 5 and SEQ ID NO: 6.

9. The process of claim 1, wherein the furanoside substrate is a natural furanoside substrate, preferably a natural furanosyl-containing polysaccharide, more preferably is arabinoxylan or arabinan, such as, for example, branched or debranched arabinan.

10. The process of claim 1, wherein the furanoside substrate is an activated furanoside donor.

11. The process of claim 1, wherein the enzyme is a mutant Araf51 enzyme and the furanoside substrate is the selection substrate of the mutant Araf51 enzyme, preferably said selection substrate is p-nitrophenyl α-L-arabinofuranoside.

12. The process of claim 1, wherein the product of interest is an alkyl-furanoside, preferably an alkyl-arabinofuranoside, more preferably the product is selected from the group comprising butyl furanoside, n-butylfuranoside, polyfuranoside, octyl-furanoside, methyl α-L-arabinofuranoside; or an alkenyl-furanoside or an allylic furanoside.

13. The process of claim 1, wherein the alcohol acceptor is an aliphatic alcohol, preferably selected from the group comprising methanol, ethanol, propanol, isopropanol, butanol, pentanol, hexanol, solketal, allylic alcohols and alkenic alcohols.

14. A mutant Araf51 enzyme showing improved transglycosylation activity in comparison with the native wild-type (wt) Araf51 enzyme, wherein said mutant enzyme presents at least one of the following features:
no inhibition in presence of alcohol acceptors;
increased kinetic conversion rate; and/or
molar conversion yield of more than 30%.

15. The mutant enzyme of claim 14, encoded by a nucleotide sequence selected from the group consisting of SEQ ID NO: 2, SEQ ID NO: 3, SEQ ID NO: 4, SEQ ID NO: 5 and SEQ ID NO: 6.

16. A method for screening mutant Araf51 enzyme showing improved transglycosylation of a selection substrate activity in comparison with the native wild-type (wt) Araf51 enzyme.
